(19) United States
(12) Patent Application Publication
(10) Pub. No.: US 2025/0325824 A1
(43) Pub. Date: Oct. 23, 2025

(54) CONNECTORS FOR AN ELECTRICAL STIMULATION SYSTEM AND METHODS OF MAKING AND USING

(71) Applicant: Boston Scientific Neuromodulation Corporation, Valencia, CA (US)

(72) Inventor: Thomas Paul Lopez, Sunland, CA (US)

(21) Appl. No.: 19/225,697

(22) Filed: Jun. 2, 2025

Related U.S. Application Data
(63) Continuation of application No. 17/888,736, filed on Aug. 16, 2022, now Pat. No. 12,343,547.
(60) Provisional application No. 63/234,884, filed on Aug. 19, 2021.

(51) Int. Cl. A61N 1/375; A61N 1/08; B23K 1/00

(52) U.S. Cl. CPC A61N 1/3752; A61N 1/08; B23K 1/0016

(57) ABSTRACT

A connector that includes contact assemblies and non-conductive stack spacers separating the contact assemblies from each other, the contact assemblies and the stack spacers defining a connector lumen configured to receive a portion of an electrical stimulation lead. The contact assemblies and stack spacers are brazed together forming a sealed connector stack that resists passage of fluid between the contact assemblies and stack spacers. Alternatively or additionally, the stack spacers are made of a non-conductive ceramic, crystalline, or glass material.

### CROSS-REFERENCE TO RELATED APPLICATIONS

This application is a continuation of U.S. patent application Ser. No. 17/888,736, filed Aug. 16, 2022, which claims the benefit under 35 U.S.C. § 119(e) of U.S. Provisional Patent Application Ser. No. 63/234,884, filed Aug. 19, 2021, both of which are incorporated herein by reference.

### FIELD

The present disclosure is directed to the area of implantable electrical stimulation systems and methods of making and using the systems. The present disclosure is also directed to connectors for an electrical stimulation system, as well as the system and methods for making and using the connectors.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator) and one or more stimulator electrodes. The one or more stimulator electrodes can be disposed along one or more leads, or along the control module, or both. The stimulator electrodes are in contact with or near the nerves, muscles, or other tissue to be stimulated. The pulse generator in the control module generates electrical pulses that are delivered by the electrodes to body tissue.

### BRIEF SUMMARY

One aspect is a connector that includes contact assemblies and non-conductive stack spacers separating the contact assemblies from each other, the contact assemblies and the stack spacers defining a connector lumen configured to receive a portion of an electrical stimulation lead, wherein the contact assemblies and stack spacers are brazed together forming a sealed connector stack that resists passage of fluid between the contact assemblies and stack spacers.

Another aspect is a connector that includes contact assemblies and non-conductive stack spacers separating the contact assemblies from each other, the contact assemblies and the stack spacers defining a connector lumen configured to receive a portion of an electrical stimulation lead, wherein the stack spacers are made of a non-conductive ceramic, crystalline, or glass material.

In at least some aspects, the stack spacers are ceramic. In at least some aspects, the connector further includes a braze material disposed between adjacent ones of the contact assemblies and the stack spacers. In at least some aspects, the connector further includes an end stop brazed to a proximal-most one of the contact assemblies. In at least some aspects, the connector further includes a flange brazed to a distal-most one of the contact assemblies.

In at least some aspects, the contact assembly includes an outer contact housing, an inner contact housing, and a connector contact. In at least some aspects, the outer contact housing is welded to the inner contact housing.

Yet another aspect is a control module that includes a sealed housing; an electronics subassembly disposed in the sealed housing; and any of the connectors described above coupled to the sealed housing, where the contact assemblies of the connector are electrically coupled to the electronics subassembly.

A further aspect is a lead extension that includes any of the connectors described.

Another aspect is a method of making a connector. The method includes forming a stack of contact assemblies and non-conductive stack spacers separating the contact assemblies from each other, wherein the contact assemblies and the stack spacers define a connector lumen configured to receive a portion of an electrical stimulation lead; and joining the contact assemblies and stack spacers to form a connector stack that resists passage of water between the contact assemblies and stack spacers.

In at least some aspects, the joining includes brazing the contact assemblies and stack spacers to form the connector stack. In at least some aspects, the forming and the joining includes flowing a glass material between the connector assemblies and hardening the glass material to form the stack spacers. In at least some aspects, the forming and the joining includes growing a crystalline material between the connector assemblies to form the stack spacers.

In at least some aspects, the method further includes attaching an end stop to a proximal-most one of the contact assemblies. In at least some aspects, the method further includes attaching a flange to a distal-most one of the contact assemblies. In at least some aspects, the method further includes coupling a retention block to the flange.

In at least some aspects, the contact assembly includes an outer contact housing, an inner contact housing, and a connector contact, wherein the forming and the joining includes forming a stack of the outer contact housings of the contact assemblies and the stack spacers separating the outer contact housings; joining the outer contact housings to the stack spacers; for each of the contact assemblies, inserting the inner contact housing and the connector contact into the outer contact housing; and physically attaching the outer contact housing to the inner contact housing. In at least some aspects, the method further includes inserting non-conductive, polymeric spacers between the connector contacts.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system;

FIG. 2 is a schematic side view of one embodiment of an electrical stimulation lead;

FIG. 3 is a schematic side view of one embodiment of a lead extension suitable for coupling with the electrical stimulation lead of FIG. 2;

FIG. 4 is a schematic side view of one embodiment of a control module suitable for receiving either the lead of FIG. 2 or the lead extension of FIG. 3;

FIG. 5A is a schematic cross-sectional view of one embodiment of a stack, including multiple contacts, for a connector assembly;

FIG. 5B is a schematic cross-sectional view of the stack of FIG. 5A with a single contact inserted into the stack;

FIG. 5C is a schematic cross-sectional view of the stack of FIG. 5A with a full set of contacts inserted into the stack;

FIG. 5D is a schematic cross-sectional view of one embodiment of connector assembly with the stack and set of contacts of FIG. 5C;

FIG. 6 is a schematic cross-sectional view of one embodiment of control module with two of the connector assemblies of FIG. 5D; and

FIG. 7 is a schematic overview of one embodiment of components of a stimulation system, including an electronic subassembly disposed within a control module, according to the invention.

### DETAILED DESCRIPTION

The present disclosure is directed to the area of implantable electrical stimulation systems and methods of making and using the systems. The present disclosure is also directed to connectors for an electrical stimulation system, as well as the system and methods for making and using the connectors.

Suitable implantable electrical stimulation systems include, but are not limited to, a least one lead with one or more electrodes disposed on a distal portion of the lead and one or more terminals disposed on one or more proximal portions of the lead. Leads include, for example, percutaneous leads, paddle leads, cuff leads, or any other arrangement of electrodes on a lead. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734;7,761,165; 7,783,359; 7,792,590; 7,809,446; 7,949,395; 7,974,706; 8,175,710; 8,224,450; 8,271,094; 8,295,944; 8,364,278; 8,391,985; and 8,688,235; and U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; and 2013/0197602, all of which are incorporated by reference. In the discussion below, a percutaneous lead will be exemplified, but it will be understood that the methods and systems described herein are also applicable to paddle leads and other leads.

The leads and electrical stimulation systems can be used for any suitable application including, but not limited to, deep brain stimulation, spinal cord stimulation, peripheral nerve stimulation, dorsal root ganglion stimulation, sacral nerve stimulation, or stimulation of other nerves, muscles, and tissues.

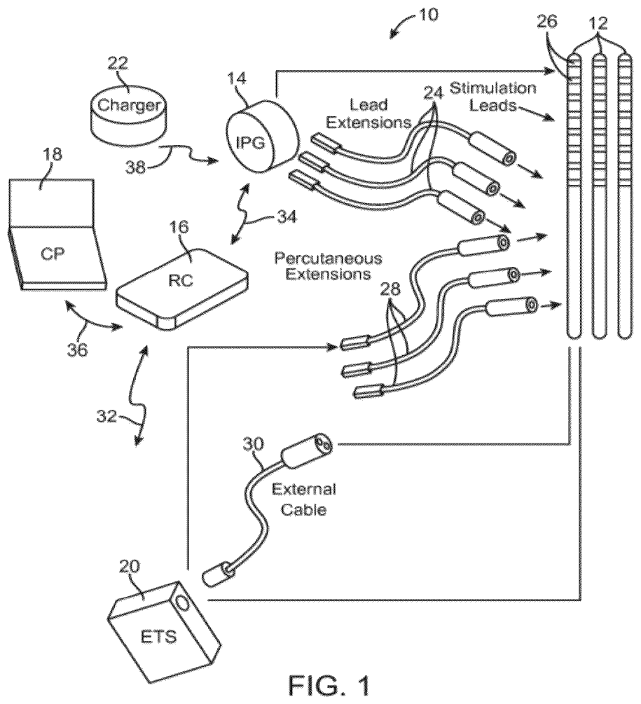

Turning to FIG. 1, one embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22.

The IPG 14 is physically connected, optionally, via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The implantable pulse generator can be implanted into a patient's body, for example, below the patient's clavicle area or within the patient's buttocks or abdominal cavity. The implantable pulse generator can have eight stimulation channels which may be independently programmable to control the magnitude of the current stimulus from each channel. In at least some embodiments, the implantable pulse generator can have more or fewer than eight stimulation channels (e.g., 4-, 6-, 16-, 32-, or more stimulation channels). The implantable pulse generator can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni-or bi-directional communications link 34. Such communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. Alternately, or additionally, stimulation parameters can be programed via wireless communications (e.g., Bluetooth) between the RC 16 (or external device such as a hand-held electronic device) and the IPG 14.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is expressly incorporated herein by reference. Other examples of electrical stimulation systems can be found at U.S. Pat. Nos. 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, as well as the other references cited above, all of which are incorporated herein by reference in their entireties.

One or more leads are configured for coupling with a control module. The term “control module” is used herein to describe a pulse generator (e.g., the IPG 14 or the ETS 20 of FIG. 1). Stimulation signals generated by the control module are emitted by electrodes of the lead(s) to stimulate patient tissue. The electrodes of the lead(s) are electrically coupled to terminals of the lead(s) that, in turn, are electrically coupleable with the control module. In some embodiments, the lead(s) couple(s) directly with the control module. In other embodiments, one or more intermediary devices (e.g., a lead extension, an adaptor, a splitter, or the like) are disposed between the lead(s) and the control module.

Percutaneous leads are described herein for clarity of illustration. It will be understood that paddle leads and cuff leads can be used in lieu of, or in addition to, percutaneous leads. The leads can include any suitable number of electrodes including, but not limited to, 4, 6, 8, 10, 12, 16, 20, 24, 30, 32, or more electrodes. The leads can include any suitable combination of ring electrodes, a distal-tip electrode, or one or more segmented electrodes. The term “elongated member” used herein includes leads (e.g., percutaneous, paddle, cuff, or the like), as well as intermediary devices (e.g., lead extensions, adaptors, splitters, or the like).

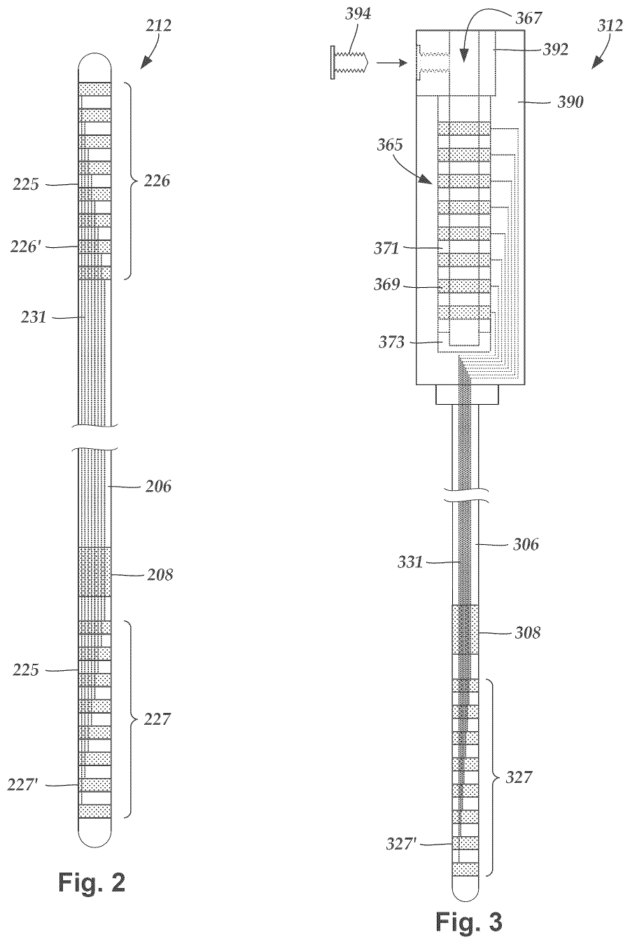

FIG. 2 shows, in schematic side view, one embodiment of a lead 212 suitable for implanting into a patient and providing electrical stimulation. In some embodiments, the lead 212 is coupled directly to a control module. In other embodiments, the lead 212 is coupled to the control module via one or more intermediary devices. In the illustrated embodiment, an array of electrodes 226, which includes electrode 226′, is disposed along a distal portion of a lead body 206 lead and an array of lead terminals 227, which includes lead terminal 227′, is disposed along a proximal portion of the lead body. Non-conductive spacers 225 separate the electrodes 226 and terminals 227 and may be part of the lead body 206 or separate elements added during construction of the lead 212. Lead conductors, such as lead conductor 231, extend along a longitudinal length of the lead and electrically couple the array of electrodes 226 to the array lead terminals 227.

Conductors can extend along the longitudinal length of the lead within one or more lumens defined in the lead. In other instances, the conductors may extend along the lead within the lead body itself. In at least some embodiments, the lead 212 includes a retention ring 208 disposed along the proximal portion of the body to facilitate coupling of the proximal portion of the lead to a connector. The connector may be disposed on or within a control module. Alternatively, the retention ring 208 can be used to facilitate coupling of the proximal portion of the lead to a connector of an intermediary device, such as a lead extension which, in turn, is coupled to a connector of a control module.

FIG. 3 shows, in schematic side view, one embodiment of a lead extension 312 suitable for implanting into a patient and coupling a lead, such as the lead 212, to a control module. The lead extension 312 includes a lead-extension body 306 having a distal portion and a proximal portion. A lead-extension connector 390 is disposed along the distal portion of the lead-extension body 306 and an array of lead-extension terminals 327, such as lead-extension terminal 327′, are disposed along the proximal portion of the lead-extension body 306.

The lead-extension connector 390 contains a lead-extension connector stack 365 that defines a connector lumen 367 configured to receive the proximal portion of an elongated member (e.g., lead 212). The lead-extension connector stack 365 includes lead-extension connector contacts, such as lead-extension connector contact 369, arranged along the connector lumen 367 and configured to electrically couple with terminals of the elongated member (e.g., lead 212) when the proximal portion of the elongated member is received by the lead-extension connector 390. The connector contacts can be electrically isolated from one another by electrically-nonconductive spacers, such as spacer 371. The connector stack may also include an end stop 373 to promote alignment of the elongated-member terminals with the lead-extension connector contacts.

The lead-extension connector 390 further includes a retention assembly for facilitating retention of the proximal portion of the elongated member (e.g., lead 212) when the proximal portion of the elongated member is received by the lead-extension connector 390. In the illustrated embodiment, the retention assembly includes a lead-extension retention block 392. The lead-extension retention block 392 is positioned to align with the retention ring (208 in FIG. 2) of the elongated member when the elongated member is received by the lead-extension connector 390. In the illustrated embodiment, the retention assembly further includes a retaining member (e.g., a set screw, a pin, or the like) 394 for pressing the retention ring of the inserted elongated member against the retention block to retain inserted elongated member within the lead-extension connector 390.

Lead-extension conductors, such as lead-extension conductor 331, extend along a longitudinal length of the lead extension and electrically couple the lead-extension connector contacts to the array of lead-extension terminals 327. The lead-extension conductors can extend along the longitudinal length of the lead-extension body within one or more lumens defined in the lead extension. In other instances, the lead-extension conductors may extend along the lead extension within the lead-extension body itself. The lead extension 312 includes an retention ring 308 disposed along the proximal portion of the lead-extension body to facilitate coupling of the proximal portion of the lead extension to a connector, such as a control-module connector, another lead-extension connector, or the like.

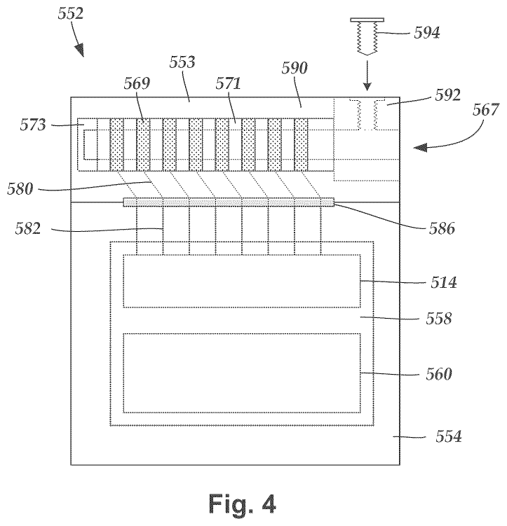

FIG. 4 shows, in schematic cross-sectional side view, a control module 552 suitable for coupling with an elongated member (e.g., the lead 212, the lead extension 312, or other intermediary device). The control module 552 includes a header 553 disposed along an outer surface of a sealed housing 554 that contains an electronic subassembly 558 with a pulse generator 514 and, optionally, a power supply 560.

A connector 590 is disposed in the header 553. The connector 590 is configured to receive an elongated device (e.g., the lead 212, the lead extension 312, or other intermediary device). The connector 590 defines a connector lumen 567 configured to receive the proximal portion of the elongated member. An array of connector contacts, such as connector contact 569, is arranged along the connector lumen 567 and configured to electrically couple with terminals of the elongated member when the proximal portion of the elongated member is received by the connector 590. The connector contacts can be electrically isolated from one another by electrically-nonconductive spacers, such as spacer 571. The connector stack may also include an end stop 573 to promote alignment of the elongated-member terminals with the connector contacts.

Wires or contacts, such as wire 582, are electrically coupled to the electrical subassembly 558 and extend within the sealed housing 554 to a feedthrough interface 586 disposed along an interface between the header 553 and the sealed housing 554. The connector contacts are electrically coupled to interconnect conductors, such as wire 580, that extend along the header 553 and electrically couple the connector contacts to the wires 582 (and possibly feedthrough pins) at the feedthrough interface 586. In some embodiments, the header 553 is positioned over the feedthrough interface 586.

The connector 590, optionally, includes a retention assembly for facilitating retention of the proximal portion of the elongated member when the proximal portion of the elongated member is received by the control module 552. In the illustrated embodiment, the retention assembly includes a retention block 592. The retention block 592 is positioned to align with a retention sleeve of the elongated member when the elongated member is received by the connector 590. In the illustrated embodiment, the retention assembly further includes a retaining member (e.g., a set screw, a pin, or the like) 594 for pressing the retention sleeve of the inserted elongated member against the retention block to retain inserted elongated member within the connector 590.

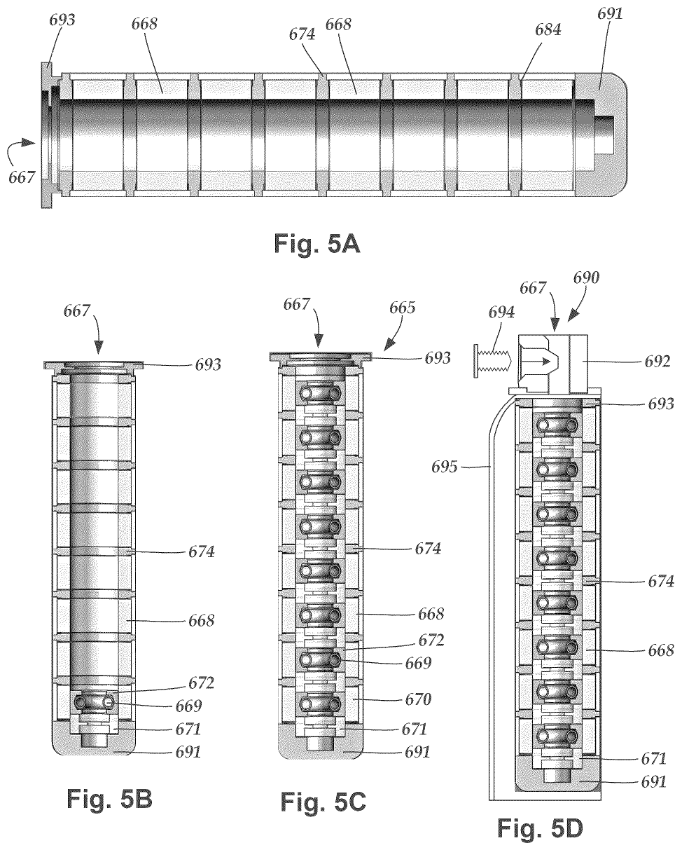

FIGS. 5A to 5D illustrate one embodiment of the manufacture of a connector 690 which can be a connector of a lead extension 312, such as lead extension connector 390, or a connector of a control module 552, such as connector 590, or any other suitable connector for receiving terminals 227, 327 of a lead 212 or lead extension 312 or the like. The connector 690 includes a connector stack 665 that defines a connector lumen 667 and includes contact assemblies 670, stack spacers 674, non-conductive spacers 671, an end stop 691, and an optional flange 693 disposed along the connector lumen 667, as illustrated in FIG. 5C.

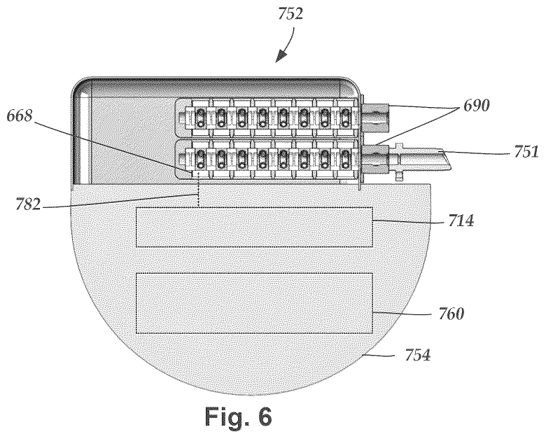

In at least some embodiments, the connector 690 can be a hermetic connector. There is interest in hermetic connectors. A hermetic connector prevents or substantially resists the flow of fluid, such as water or bodily fluids, from the connector lumen 667 through, or between, the contact assemblies 670 and stack spacers 674 to enter, for example, the header 553 (FIG. 4) (or sealed housing 554 (FIG. 4) when the connector extends into the sealed housing instead of the header 553—see, FIG. 6.) In at least some embodiments, a hermetic connector can be disposed in the sealed housing 554 to eliminate a feedthrough interface 586 and separate header 553, as illustrated, for example, in FIG. 6.

In at least some embodiments, the stack spacers 674 can be made of any suitable non-conductive ceramic, crystalline, or glass material including, but not limited to, aluminum oxide (including, but not limited to, alumina or crystalline aluminum oxide such as corundum, ruby, or sapphire), glass, or the like or any combination thereof. The stack spacers 674 can be formed by any suitable method including, but not limited to, molding (for example, by molding a ceramic material or by melting glass and pouring into a mold), crystal growth, or the like or any combination thereof. In at least some embodiments, the end stop 691 is also made of the same material as the stack spacers 674.

In at least some embodiments, the contact assembly 670 includes a connector contact 669, an inner contact housing 672, and an outer contact housing 668, as illustrated in FIG. 5B. The inner contact housing 672 holds the contact 669 in position along the connector lumen 667 in order to make contact with terminals 227, 327 of a lead 212 or lead extension 312. The outer contact housing 668 can be separate element to facilitate construction of the connector stack 665 as described below. In at least some embodiments, the inner contact housing 672 and outer contact housing 668 are a single element (for example, a single contact housing) or are both absent from the contact assembly 670.

In at least some embodiments, the outer contact housings 668 (or the contact assemblies 670) and stack spacers 674 are arranged in an alternating pattern. The outer contact housings 668 (or the contact assemblies 670), stack spacers 674, end stop 691, and, optionally, the flange 693 are joined together, as illustrated in FIG. 5A. In at least some embodiments, this joining forms a sealed connector stack that resists the passage of water and bodily fluids (and, at least in some embodiments, other fluids, such as helium or other gases) between the contact housings 668 (or the contact assemblies 670) and stack spacers 674.

In at least some embodiments, the outer contact housings 668 (or the contact assemblies 670), stack spacers 674, end stop 691, and, optionally, the flange 693 are joined together by brazing. In at least some embodiments, the brazing of the outer contact housings 668 (or contact assemblies 670), stack spacers 674, end stop 691, and, optionally, the flange 693 forms a hermetic structure that prevents or resists passage of fluid, such as water, bodily fluids, or the like through the hermetic structure. In at least some embodiments, the hermetic structure prevents or resists passage of helium or other gas through the hermetic structure. In at least some embodiments, to facilitate brazing, the connector stack 665 includes a braze material 694 selected to braze the outer contact housings 668 (or contact assemblies 670) to the stack spacers 674. For example, the braze material 694 can be gold, or the like or any combination thereof. In at least some embodiments, the braze material 694 is selected based on the materials of the outer contact housings 668 and stack spacers 674.

Other methods of joining the outer contact housings 668 (or the contact assemblies 670) and stack spacers 674 (and optionally one or both of the end stop 691 and the flange 693). For example, one method includes flowing or otherwise disposing a glass material between the outer contact housings (or the contact assemblies 670) and solidifying or hardening the glass material to form the stack spacers 674. Another method includes growing a crystalline material between the outer contact housings (or the contact assemblies 670) to form the stack spacers 674. Yet other methods forming an alternating stack of the outer contact housings 668 (or the contact assemblies 670) and stack spacers 674 and applying pressure on the stack to join the outer contact housings 668 (or the contact assemblies 670) and stack spacers 674. Any other suitable method for joining or forming the stack of outer contact housings 668 (or the contact assemblies 670) and stack spacers 674 can be used.

In at least some embodiments, as illustrated in FIGS. 5B and 5C, subsequently the non-conductive spacers (or seals) 671 and connector contacts 669 (within the inner contact housing 672) are alternatingly inserted into the connector lumen 667. The non-conductive spacers 671 separate and electrically isolate the connector contacts 669 from each other.

In at least some embodiments, each of the contact housings 672 of each of the connector contacts 669 is electrically coupled to a corresponding one of the outer contact housing 668 (for example, physically coupled by seam or spot welding using a laser or the like, soldering, or otherwise forming a physical attachment of the inner contact housing 672 or connector contact 669 to the outer contact housing 668) to form the contact assembly 670 after the connector contact 669 is placed. Other methods for electrically coupling the connector contact 669 (within the inner contact housing 672) to the outer contact housing 668 can be used including, but not limited to, passive coupling through contact between the inner contact housing 672 and the outer contact housing 668.

In an alternative method of manufacture, instead of just the outer contact housing 668, the entire contact assembly 670 can be stacked and brazed with the stack spacers 674. Then, non-conductive spacers 671 can be formed between the connector contacts 669 by injecting a non-conductive material such as a polymeric material (for example, silicone) or a polymer precursor into the connector lumen 667 and then cooling, crosslinking, or otherwise modifying the non-conductive material to form the spacers 671. In at least some embodiments, a mandrel can be inserted into the connector lumen 667 after injecting the non-conductive material, and, optionally, prior to forming the spacers 671, to facilitate clearing the connector lumen of the non-conductive material.

In FIG. 5D, the connector stack 665 is welded into a case 695 and the retention block 692 (with fastener 694) is coupled to the flange 693 or connector stack 665 to produce the connector 690. In at least some embodiments, the connector 690 provides a hermetic seal against the passage of fluid, such as water, bodily fluids, helium or other gas, or the like.

In at least some embodiments, as illustrated in FIG. 6, a hermetic connector 690 can be part of a control module 752 that does not include a header 553 (FIG. 4) or feedthrough interface 586 (FIG. 4). (The header 553 and feedthrough interface 586 are typically provided to couple non-hermetic connectors 590 to a hermetically sealed housing 554.) The control module 752 can include a sealed housing 754 with one or more of the hermitic connectors 690 extending into the sealed housing. The control module 752 can include a pulse generator 714 and optional power source 760. The pulse generator can be coupled to the contacts 669 by wires 782.

In at least some embodiments, a strain relief component 751 can extend from the connector 690, as illustrated in FIG. 6. For example, the strain relief component 751 can be made of silicone or other flexible material. In at least some embodiments, for a control module 752 with multiple connectors 690, an individual strain relief component 751 can extend from each connector 690. These strain relief components 751 can be separate from each other or can be connected to one another (or to a subset of each other).

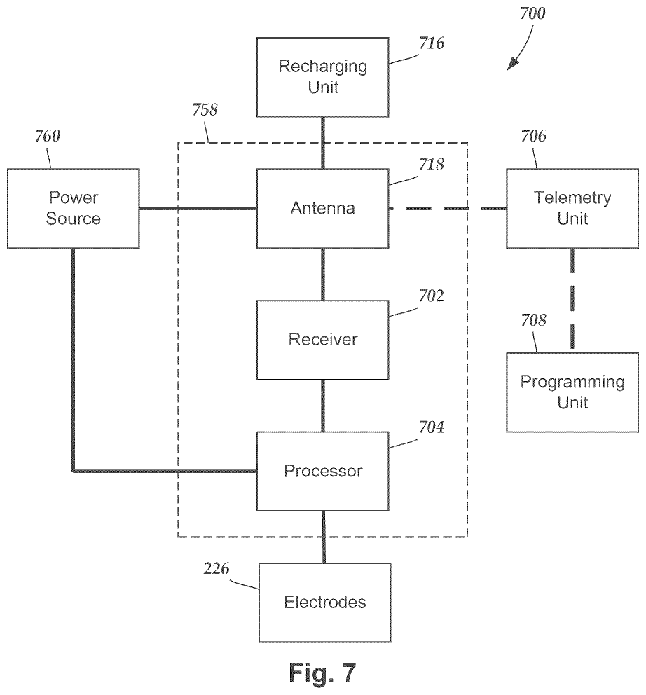

FIG. 7 is a schematic overview of one embodiment of components of an electrical stimulation system 700 including an electronic subassembly 758 disposed within a control module. The electronic subassembly 758 may include one or more components of the IPG. It will be understood that the electrical stimulation system can include more, fewer, or different components and can have a variety of different configurations including those configurations disclosed in the stimulator references cited herein.

Some of the components (for example, a power source 760, an antenna 718, a receiver 702, and a processor 704) of the electrical stimulation system can be positioned on one or more circuit boards or similar carriers within a sealed housing of an implantable pulse generator (see e.g., 14 in FIG. 1), if desired. Any power source 760 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 718 or a secondary antenna. The external power source can be in a device that is mounted on the skin of the user or in a unit that is provided near the user on a permanent or periodic basis.

If the power source 760 is a rechargeable battery, the battery may be recharged using the optional antenna 718, if desired. Power can be provided to the battery for recharging by inductively coupling the battery through the antenna to a recharging unit 716 external to the user. Examples of such arrangements can be found in the references identified above. The electronic subassembly 758 and, optionally, the power source 760 can be disposed within a control module (e.g., the IPG 14 or the ETS 20 of FIG. 1).

In one embodiment, electrical stimulation signals are emitted by the electrodes (e.g., 26 in FIG. 1) to stimulate nerve fibers, muscle fibers, or other body tissues near the electrical stimulation system. The processor 704 is generally included to control the timing and electrical characteristics of the electrical stimulation system. For example, the processor 704 can, if desired, control one or more of the timing, frequency, strength, duration, and waveform of the pulses. In addition, the processor 704 can select which electrodes can be used to provide stimulation, if desired. In some embodiments, the processor 704 selects which electrode(s) are cathodes and which electrode(s) are anodes. In some embodiments, the processor 704 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 708 that, for example, allows modification of pulse characteristics. In the illustrated embodiment, the processor 704 is coupled to a receiver 702 which, in turn, is coupled to the optional antenna 718. This allows the processor 704 to receive instructions from an external source to, for example, direct the pulse characteristics and the selection of electrodes, if desired.

In one embodiment, the antenna 718 is capable of receiving signals (e.g., RF signals) from an external telemetry unit 706 which is programmed by the programming unit 708. The programming unit 708 can be external to, or part of, the telemetry unit 706. The telemetry unit 706 can be a device that is worn on the skin of the user or can be carried by the user and can have a form similar to a pager, cellular phone, or remote control, if desired. As another alternative, the telemetry unit 706 may not be worn or carried by the user but may only be available at a home station or at a clinician's office. The programming unit 708 can be any unit that can provide information to the telemetry unit 706 for transmission to the electrical stimulation system 700. The programming unit 708 can be part of the telemetry unit 706 or can provide signals or information to the telemetry unit 706 via a wireless or wired connection. One example of a suitable programming unit is a computer operated by the user or clinician to send signals to the telemetry unit 706.

The signals sent to the processor 704 via the antenna 718 and the receiver 1302 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 700 to cease operation, to start operation, to start charging the battery, or to stop charging the battery. In other embodiments, the stimulation system does not include the antenna 718 or receiver 702 and the processor 704 operates as programmed.

Optionally, the electrical stimulation system 700 may include a transmitter (not shown) coupled to the processor 704 and the antenna 718 for transmitting signals back to the telemetry unit 706 or another unit capable of receiving the signals. For example, the electrical stimulation system 700 may transmit signals indicating whether the electrical stimulation system 700 is operating properly or not or indicating when the battery needs to be charged or the level of charge remaining in the battery. The processor 704 may also be capable of transmitting information about the pulse characteristics so that a user or clinician can determine or verify the characteristics.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. A connector, comprising:
a plurality of contact assemblies, wherein each of the contact assemblies comprises an outer contact housing, a separate inner contact housing attached to the outer contact housing, and a connector contact, wherein the outer contact housing, inner contact housing and connector contact are each conductive; and
a plurality of non-conductive stack spacers separating the contact assemblies from each other, the contact assemblies and the stack spacers forming a connector stack defining a connector lumen configured to receive a portion of an electrical stimulation lead, wherein the contact assemblies and stack spacers are brazed together forming a sealed connector stack that resists passage of fluid between the contact assemblies and stack spacers, wherein the inner contact housing of each of the contact assemblies defines a groove within which the connector contact resides and two separate interior surfaces that are exposed within the connector lumen and separated by the groove and the connector contact.

2. The connector of claim 1, wherein the stack spacers are ceramic.

3. The connector of claim 1, further comprising a braze material disposed between adjacent ones of the contact assemblies and the stack spacers.

4. The connector of claim 1, further comprising an end stop brazed to a proximal-most one of the contact assemblies.

5. The connector of claim 1, further comprising a flange brazed to a distal-most one of the contact assemblies.

6. The connector of claim 1, wherein the outer contact housing is welded to the inner contact housing.

7. The connector of claim 1, wherein the outer contact housing and the stack spacers have a same inner diameter.

8. The connector of claim 7, wherein the inner diameter of the outer contact housing is greater than or equal to an outer diameter of the inner contact housing.

9. The connector of claim 1, further comprising a plurality of non-conductive, polymeric spacers disposed radially inward of the stack spacers and separating the contact assemblies from each other.

10. The connector of claim 1, wherein, with respect to the contact assemblies, the stack spacers only engage the outer contact housing.

11. A control module, comprising:
a sealed housing;
an electronics subassembly disposed in the sealed housing; and
the connector of claim 1 coupled to the sealed housing, wherein the contact assemblies of the connector are electrically coupled to the electronics subassembly.

12. A method of making the connector of claim 1, the method comprising:
forming the connector stack of the plurality of contact assemblies and the plurality of stack spacers separating the contact assemblies from each other; and
joining the contact assemblies and stack spacers to resist passage of water between the contact assemblies and stack spacers.

13. A method of making a connector, the method comprising:
providing a plurality of contact assemblies; and
growing a crystalline material between each adjacent pair of the contact assemblies to form a plurality of stack spacers, wherein the contact assemblies and the stack spacers define a connector lumen configured to receive a portion of an electrical stimulation lead and the contact assemblies and stack spacers form a connector stack that resists passage of water between the contact assemblies and stack spacers.

14. The method of claim 13, wherein the crystalline material comprises corundum, sapphire, or ruby.

15. The method of claim 13, wherein the contact assembly comprises an outer contact housing, an inner contact housing, and a connector contact, the method further comprising:
for each of the contact assemblies, inserting the inner contact housing and the connector contact into the outer contact housing; and
physically attaching the outer contact housing to the inner contact housing.

16. The method of claim 15, wherein the inner contact housing of each of the contact assemblies defines a groove within which the connector contact resides and two separate interior surfaces that are exposed within the connector lumen and separated by the groove and the connector contact.

17. A method of making a connector, the method comprising:
providing a plurality of contact assemblies; and
flowing a glass material between the contact assemblies and hardening the glass material to form a plurality of stack spacers, wherein the contact assemblies and the stack spacers define a connector lumen configured to receive a portion of an electrical stimulation lead and the contact assemblies and stack spacers form a connector stack that resists passage of water between the contact assemblies and stack spacers.

18. The method of claim 17, wherein flowing the glass material comprises melting the glass material and flowing the melted glass material between the contact assemblies.

19. The method of claim 17, wherein the contact assembly comprises an outer contact housing, an inner contact housing, and a connector contact, the method further comprising:
for each of the contact assemblies, inserting the inner contact housing and the connector contact into the outer contact housing; and
physically attaching the outer contact housing to the inner contact housing.

20. The method of claim 17, further comprising attaching an end stop to a proximal-most one of the contact assemblies.
